(19) United States
(12) Patent Application Publication
(10) Pub. No.: US 2011/0195509 A1
(43) Pub. Date: Aug. 11, 2011

(54) TREATMENT OF TH17-MEDIATED AUTOIMMUNE DISEASE VIA INHIBITION OF STAT 3

(76) Inventors: Drew Pardoll, Brookeville, MO (US); Charles Drake, Baltimore, MD (US); Timothy Harris, Baltimore, MD (US); Hua Yu, Duarte, CA (US)

(21) Appl. No.: 12/677,094

(22) PCT Filed: Sep. 5, 2008
(86) PCT No.: PCT/US08/75485

§ 371 Date: Apr. 25, 2011

Related U.S. Application Data
(60) Provisional application No. 60/967,671, filed on Sep. 6, 2007.

(51) Int. Cl. G01N 33/68; C07H 21/02; C07K 16/18; C07K 16/24; G01N 33/48

(52) U.S. Cl. 436/86; 536/24.5; 530/389.1; 530/389.2; 436/94

(57) ABSTRACT

Methods for treating autoimmune disease using one or more inhibitor of STAT3 are provided herein. Also disclosed are methods for diagnosing and monitoring autoimmune disease or the propensity to develop autoimmune disease in a subject. The present invention demonstrates that inhibition of STAT3 prevents development of autoimmune disease in vivo. Based on this finding, Stat3 inhibitors can be used to treat and/or diagnose autoimmune disease in a subject.

### CROSS-REFERENCE TO RELATED APPLICATION

This application claims the benefit of U.S. Provisional Patent Application Ser. No. 60/967,671, filed on Sep. 6, 2007, which is incorporated herein by reference.

### BACKGROUND

STAT3 has emerged as a potentially important transcription factor in a number of autoimmune diseases. Initially, analysis of leukocytes in inflammatory synovial fluid from arthritic joints demonstrated activation of STAT3 by IL-6 (1). STAT3 activation in the brain was demonstrated during the acute phase of experimental autoimmune encephalomyelitis (EAE) (2). STAT3 activation has also been described in peripheral blood mononuclear cells derived from patients with multiple sclerosis (3). Finally, STAT3 activation has been described in patients with systemic lupus erythematosus (4).

Until recently, cell-mediated autoimmunity was believed to be mediated in many systems by T_{H}1 CD4 T cells, characterized by secretion of the cytokine IFN-γ and activation of cytotoxic CD8 T cells. This concept was supported by p40 knockout (KO) mice that lack expression of the cytokine IL-12 (5); however, IL-12 is a heterodimeric cytokine that is composed of an alpha (p35) subunit and a beta (p40) subunit. Recently, IL-23 was demonstrated to also use an identical p40 subunit, but in this case paired with a unique p19 subunit (6). IL-23 drives a unique CD4 T cell differentiation pathway termed T_{H}17 (7, 8), characterized by the production of IL-17 rather than the T_{H}1-defining cytokine, IFN-γ, or the T_{H}2 defining cytokine, IL-4. Commitment to the T_{H}17 pathway requires the presence of both IL-6 and TGF-β during in vitro culture conditions in which naïve T cells are activated through their T cell receptor (9, 10).

STAT3 activation has been observed in several autoimmune diseases, suggesting that STAT3-mediated pathways promote pathologic immune responses. The fundamental role of STAT3 signaling in autoimmunity relates to its absolute requirement for generating T_{H}17 T cell responses. STAT3 is a master regulator of this pathogenic T cell subtype, acting at multiple levels in vivo, including T_{H}17 T cell differentiation and cytokine production, as well as induction of RORγt and the IL-23 receptor. Neither naturally occurring T_{H}17 cells nor T_{H}17-dependent autoimmunity occurs when STAT3 is ablated in CD4 cells. Furthermore, ablation of STAT3 signaling in CD4 cells results in increased T_{H}1 responses, indicating that STAT3 signaling skews T_{H} responses away from the T_{H}1 pathway and toward the T_{H}17 pathway. STAT3 is a candidate target for T_{H}17-dependent autoimmune disease immunotherapy that could selectively inhibit pathogenic immune pathways.

All references cited herein are hereby incorporated by reference in their entirety.

### SUMMARY

In the present invention, T cell targeted STAT3 KO is utilized to show for the first time that STAT3 signaling is required for T_{H}17 T cell differentiation in vivo in a number of autoimmune models. In addition, STAT3 is required for the maintenance of endogenous, gut-resident, T_{H}17 cells. In these models, promotion of IL-23/T_{H}17 immunity by STAT3 occurs at the expense of IL-12/T_{H}1 immunity. Message-level expression of the transcription factor RORγt appears to occur downstream of STAT3 signaling, as this expression is virtually absent in CD4 T cells that lack STAT3 signaling (14). Taken together, these findings demonstrate that STAT3 signaling is a central component of T_{H}17-dependent autoimmune processes and is a target for therapeutic intervention in autoimmune disease.

The invention provides for compositions and methods for treating an autoimmune disease by inhibition of STAT3. In one aspect, the invention provides compositions and methods for treating an autoimmune disease by use of agents that inhibit or block STAT3 activity or STAT3 signaling.

In another aspect, the invention provides for methods for treating an autoimmune disease in an individual by administering to the individual an agent that reduces, inhibits or blocks STAT3 activity in an amount effective to reduce, inhibit or block the activity of STAT3. In one embodiment, the autoimmune disease is associated with inflammation. In another embodiment, the disease is selected from the group consisting of arthritis, multiple sclerosis, rheumatoid arthritis, Crohn's disease, bacterially induced colitis, lupus, diabetes, inflammatory bowel disease, scleroderma, uveitis, vasculitis, psoriasis, osteoporosis, asthma, bronchitis, allergic rhinitis, chronic obstructive pulmonary disease, artherosclerosis, and septic shock. In another embodiment, the disease is an inflammatory disease or condition that involves any organ or tissue containing cells in which the presence and/or expression of RORγt has been shown.

In another aspect, the invention provides for methods for treating a condition associated with an autoimmune disease by administering to the individual an effective amount of an antagonist to STAT3 such that STAT3 activity is antagonized, wherein the antagonist is selected from the group consisting of an antibody, a small molecule, an antisense RNA, RNAi, siRNA or shRNA, siRNA or shRNA conjugated to ligands that specifically target T cells, antibodies to T cell surface molecules, peptides specific for T cell surface molecules, RNA aptamers specific for T cell surface molecules, and T cell tropic gene delivery vectors encoding STAT3 siRNA or shRNA.

In another aspect, the invention provides for compositions comprising an antagonist to STAT3 wherein the antagonist is capable of modulating the activity of STAT3 in an individual. In one embodiment, the activity of STAT3 includes STAT3 signaling and its downstream effects.

In another aspect, the invention provides for methods for ameliorating the symptoms associated with autoimmune disease comprising administering to an individual suffering from an autoimmune disease, a sufficient amount of an antagonist to STAT3 to inhibit or decrease the induction of T_{H}17 cells to the extent that would otherwise give rise to an autoimmune response.

In another aspect, the invention provides for a method of diagnosing a subject as having or having a propensity to develop an autoimmune disease comprising determining the level or biological activity of a STAT3 polypeptide or nucleic acid in a sample from the subject, wherein a greater level or biological activity of the STAT3 polypeptide or nucleic acid in the sample relative to a reference sample or reference level is diagnostic of an autoimmune disease or a propensity to develop an autoimmune disease in the subject.

In another aspect, the invention provides for a non-human animal for use as a model of autoimmune disease. The non-human animal is one whose genome comprises a conditional deletion of STAT3 expression in its CD4 population of T-cells. Such an animal is particularly useful for use as a model of T_{H}17-mediated autoimmune disease.

### BRIEF DESCRIPTION OF THE DRAWINGS

FIG. 1. Conditional deletion of STAT3 in CD4 lymphocytes. Naïve CD4 T cells from WT and CD4 STAT3^{-/-} (CD4-Cre×STAT3flox) mice were activated in vitro with PMA and ionomycin in the presence of IL-6, TGFβ, IL-12, or IL-6+TGFβ. (a) intracellular staining for phospho-STAT3 in unstimulated WT cells (dotted line), WT cells activated in the presence of IL-6 (black line), and CD4 STAT3^{-/-} cells activated in the presence of IL-6 (gray line). (b) Nuclear extracts analyzed by EMSA+/−STAT supershift. Activation: −=No, +=Yes. Cytokines: N=none, β=TGFβ, 12=IL-12, 6=IL-6. Supershift: N=None, 1=STAT1, 3=STAT3. (c) Naïve (CD62L⁺) CD4 T cells from WT (top panels) and CD4 STAT3^{-/-} (bottom panels) mice were activated in vitro with anti-CD3/CD28 microbeads in the presence of IL-12 (middle panels) or TGFβ and IL-6 (right panels). The effector cytokines IL-17 and IFNγ were analyzed by intracellular flow cytometry following restimulation. Unactivated controls (left panels) were also analyzed.

FIG. 2. STAT3 is required in vivo for development of endogenous gut-associated T_{H}17 cells. Lymphocytes from indicated tissues were harvested from WT or CD4 STAT3^{-/-} mice and activated in vitro 4 hours followed by intracellular cytokine staining. (a) IL-17 versus IFNγ (b) IL-4 versus IFNγ.

FIG. 3. STAT3 expression in CD4 T cells is required for EAE induction. EAE was induced in WT or CD4 STAT3^{-/-} mice by immunization with MOG peptide and CFA. On day 22, lymphocytes were harvested from CNS (spinal cord), draining lymph node (LN), and spleen. (a) Intracellular staining of CD4 lymphocytes for IL-17 and IFNγ. (b) frequency of T_{H}17 and T_{H}1 cells infiltrating CNS (left) and LN (right) (c) absolute number infiltrating CNS, LN (d) Clinical disease score (e) percent of weight change. ***p<0.0001, **p=0.0182. For WT mice N=9, for CD4 STAT3^{-/-} mice N=5.

FIG. 4. STAT3 is required for the development of T_{H}17 cells in an autoimmune pneumonitis model. Naïve HA-specific Thy1.1⁺ TCR transgenic CD4 T cells from either STAT3 WT or CD4 STAT3^{-/-} backgrounds were transferred into Thy1.2⁺ C3HA recipients expressing HA in the lungs. Lung infiltrating lymphocytes were harvested on day 4. In addition, naïve HA-specific Thy1.1⁺ TCR transgenic CD4 T cells from either STAT3 WT or CD4 STAT3^{-/-} backgrounds were transferred to non-transgenic (B10.D2) hosts and infected with WT vaccinia (Vacc) or recombinant vaccinia expressing HA (Vacc-HA). Donor cells (CD4⁺, Thy1.1⁺) were analyzed for: (a) phospho-STAT3 expression and (b-e) cytokine expression by intracellular staining (ICS). (b) FACS analysis of lung-infiltrating CD4 T cells. (c) Summary of percent of lung infiltrating donor-derived T_{H}1 and T_{H}17 cells, N=5 per group, Median+/−SEM (d) Absolute numbers of lung-infiltrating donor-derived T_{H}1 and T_{H}17 cells. (e-f) Analysis of lung-infiltrating donor-derived T_{H}2 and Treg cells by intracellular staining for IL4 and FoxP3 respectively. (e) Representative FACS analysis and (f) Summary data, N=5 animals/group, median+/−SEM (g) RT-PCR analysis of lung-infiltrating donor-derived cells, recovered by high-purity (>95%) FACS sort, for cytokines, receptors and transcription factors specific for T_{H}1, T_{H}2, T_{H}17 and T_{REG} cells. Reactions performed in triplicate, N=5 pooled animals/group. Mean+/−SEM.

FIG. 5. CD4 T cell expression of STAT3 is required for the development of fatal autoimmune pneumonitis. (a) A lethal dose of HA-specific WT or CD4 STAT3^{-/-} cells were adoptively transferred into C3HA mice. Data are representative of three independent experiments. N=9 per group, ***p<0.0001(b) C3HA mice adoptively transferred with a lethal dose of HA-specific WT CD4 cells (as in a) were treated with a combination of anti-IL23R and anti-IL17 or isotype controls, N=5 animals/group, **p=0.07.

### DETAILED DESCRIPTION

The invention provides for treatment of autoimmune disease and other inflammatory conditions that involve T_{H}17 responses by use of STAT3 inhibitors and/or STAT3 antagonists. Autoimmune diseases can be modulated by using a STAT3 inhibitor. Autoimmune diseases can also be modulated by using an agent which is antagonist for STAT3. Autoimmune diseases can also be modulated by using an amount of an agent which is an inhibitor or antagonist of STAT3 that is sufficient to effect a reduction in the number and/or the functionality of T_{H}17 cells.

The present invention demonstrates that STAT3 functions as a required signal in T_{H}17 differentiation in vivo. Accordingly, STAT3 is a key signal for Th17-dependent autoimmune diseases. Additionally, the present invention demonstrates that STAT3 may mediate a relative inhibition of T_{H}1 differentiation. STAT3 plays a broad role in T_{H} skewing via reciprocal regulation of master transcription factors for the T_{H}1 and T_{H}17 lineages (T-bet and RORγt, respectively). Current therapies for autoimmune disease are limited by nonspecific immunosuppression. The present invention demonstrates that STAT3 is a key regulator of immune homeostasis and thus serves as a novel target for treatment of T_{H}17 dependent autoimmune diseases. Thus, inhibiting or antagonizing the expression of STAT3 or neutralizing its activity can modulate an inflammatory condition such as a condition associated with or resulting from T_{H}17 dependent autoimmunity.

The practice of the present invention includes use of conventional techniques of molecular biology such as recombinant DNA, microbiology, cell biology, biochemistry, nucleic acid chemistry, and immunology as described for example in: Molecular Cloning: A Laboratory Manual, second edition (Sambrook et al., 1989) and Molecular Cloning: A Laboratory Manual, third edition (Sambrook and Russel, 2001), jointly and individually referred to herein as “Sambrook”); Oligonucleotide Synthesis (M. J. Gait, ed., 1984); Animal Cell Culture (R. I. Freshney, ed., 1987); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987, including supplements through 2001); PCR: The Polymerase Chain Reaction, (Mullis et al., eds., 1994); Current Protocols in Immunology (J. E. Coligan et al., eds., 1991); The Immunoassay Handbook (D. Wild, ed., Stockton Press NY, 1994); Bioconjugate Techniques (Greg T. Hermanson, ed., Academic Press, 1996); Methods of Immunological Analysis (R. Masseyeff, W. H. Albert, and N. A. Staines, eds., Weinheim: VCH Verlags gesellschaft mbH, 1993), Harlow and Lane (1988) Antibodies, A Laboratory Manual, Cold Spring Harbor Publications, New York, and Harlow and Lane (1999) Using Antibodies: A Laboratory Manual Cold Spring Harbor Laboratory Press, Cold Spring Harbor, N.Y. jointly and individually referred to herein as “Harlow and Lane”), Beaucage et al. eds., Current Protocols in Nucleic Acid Chemistry (John Wiley & Sons, Inc., New York, 2000); and Agrawal, ed., Protocols for Oligonucleotides and Analogs, Synthesis and Properties (Humana Press Inc., New Jersey, 1993).

An “antagonist” or “inhibitor” refers to any substance, molecule, compound or agent that blocks, suppresses or reduces STAT3 expression or STAT3 activity (including STAT3 biological activity), including downstream pathways mediated by STAT3 signaling, such as signaling in T_{H}17 cell differentiation, inhibition of T_{H}1 differentiation, and/or elicitation of a cytokine expression. An “antagonist” or “inhibitor” includes an agent such as a small molecule, protein, peptide or nucleic acid molecule such as an antisense nucleic acid or a small interfering RNA molecule (siRNA or shRNA) or an antibody that prevents the expression and/or function of a designated molecule, such as STAT3.

The term “antagonist” or “inhibitor” does not imply a specific mechanism of biological action. Indeed, the term “antagonist” or “inhibitor” as used herein expressly includes and encompasses all possible pharmacological, physiological, and biochemical interactions with STAT3 whether direct or indirect, or whether interacting with STAT3 gene expression, or through another mechanism. Exemplary STAT3 antagonists include, but are not limited to, an anti-STAT3 antibody, an anti-sense molecule directed to an STAT3 (including an anti-sense molecule directed to a nucleic acid encoding STAT3), an STAT3 inhibitory compound, an STAT3 structural analog.

An “effective amount” of a substance is that amount sufficient to effect beneficial or desired results, including clinical results, and, as such, an “effective amount” depends upon the context in which it is being applied. In the context of administering a composition that modulates an autoimmune response, an effective amount of an agent which is an inhibitor of STAT3 is an amount sufficient to achieve such a modulation as compared to the response obtained when there is no inhibitor administered. An effective amount can confer immediate, short term or long term benefits of disease modification, such as suppression and/or inhibition of T_{H}17 cells or T_{H}17 differentiation. An effective amount can be administered in one or more administrations. A “therapeutically effective amount” as used herein, is intended to mean an amount sufficient to reduce by at least 10%, preferably at least 25%, more preferably at least 50%, and most preferably an amount that is sufficient to cause an improvement in one or more clinically significant symptoms in the patient.

“Treatment” or “treating” refers to therapy, prevention and prophylaxis and particularly refers to the administration of medicine or the performance of medical procedures with respect to a patient, for either prophylaxis (prevention) or to reduce the extent of or likelihood of occurrence of the infirmity or malady or condition or event in the instance where the patient is afflicted. It also refers to reduction in the severity of one or more symptoms associated with the disease or condition. In the present application, it may refer to amelioration of one or more of the following: pain, swelling, redness or inflammation associated with an inflammatory condition or an autoimmune disease. As used herein, and as well-understood in the art, “treatment” is an approach for obtaining beneficial or desired results, including clinical results. For purposes of this invention, beneficial or desired clinical results include, but are not limited to, alleviation or amelioration of one or more symptoms, diminishment of extent of disease, stabilized (i.e., not worsening) state of disease, delay or slowing of disease progression, and/or amelioration or palliation of the disease state. “Treatment” can also mean prolonging survival as compared to expected survival if not receiving treatment.

As used herein a “reference sample” is meant any sample, standard, standard curve, or level that is used for comparison purposes. A “normal reference sample” can be, for example, a prior sample taken from the same subject; a normal healthy subject; a sample from a subject not having an autoimmune disease or an inflammatory disorder; a subject that is diagnosed with a propensity to develop an autoimmune disease but does not yet show symptoms of the disease; a subject that has been treated for an autoimmune disease; or a sample of a purified reference polypeptide or nucleic acid molecule of the invention (e.g., STAT3) at a known normal concentration. By “reference standard or level” is meant a value or number derived from a reference sample. A normal reference standard or level can be a value or number derived from a normal subject who does not have an autoimmune disease. In one embodiment, the reference sample, standard, or level is matched to the sample subject by at least one of the following criteria: age, weight, body mass index (BMI), disease stage, and overall health. A standard curve of levels of purified DNA, RNA or mRNA within the normal reference range can also be used as a reference. A standard curve of levels of purified protein within the normal reference range can also be used as a reference.

As used herein, a “positive reference” is meant a biological sample, for example, a biological fluid (e.g., urine, blood, serum, plasma, or cerebrospinal fluid), tissue (e.g., vascular tissue or endothelial tissue), or cell (e.g., a vascular endothelial cell), collected from a subject who has an autoimmune disease or a propensity to develop an autoimmune disease. In addition, a positive reference may be derived from a subject that is known to have an autoimmune disease, that is matched to the sample subject by at least one of the following criteria: age, weight, BMI, disease stage, overall health, prior diagnosis of an autoimmune disease, or a family history of an autoimmune disease. A positive reference as used herein may also be a purified polypeptide or nucleic acid of the invention (e.g., recombinant or non-recombinant STAT3), or any biological sample (e.g., a biological fluid, tissue, or cell) that contains a polypeptide or nucleic acid of the invention. A standard curve of levels of purified protein, nucleic acid, or antibody for any of the polypeptides of the invention within a positive reference range can also be used as a reference.

As used herein, a “sample” is meant any bodily fluid (e.g., urine, blood, serum, plasma, or cerebrospinal fluid), tissue (e.g., cardiac tissue or endothelial tissue), or cell (e.g., endothelial cell) in which a polypeptide or nucleic acid molecule of the invention is detectable.

As used herein, a “small RNA” is meant any RNA molecule, either single-stranded or double-stranded” that is at least 15 nucleotides, preferably, 17, 18, 19, 20, 21, 22, 23, 24, 25, 26, 27, 28, 29, 30, 31, 32, 33, 34, or 35, nucleotides in length and even up to 50 or 100 nucleotides in length (inclusive of all integers in between). Preferably, the small RNA is capable of mediating RNAi. As used herein the phrase “mediates RNAi” refers to the ability to distinguish which RNAs are to be degraded by the RNAi machinery or process. Included within the term small RNA are “small interfering RNAs” and “microRNA.” In general, microRNAs (miRNAs) are small (e.g., 17-26 nucleotides), single-stranded noncoding RNAs that are processed from approximately 70 nucleotide hairpin precursor RNAs by Dicer. Small interfering RNAs (siRNAs) are of a similar size and are also non-coding, however, siRNAs are processed from long dsRNAs and are usually double stranded. siRNAs can also include short hairpin RNAs in which both strands of an siRNA duplex are included within a single RNA molecule. Small RNAs can be used to describe both types of RNA. These terms include double-stranded RNA, single-stranded RNA, isolated RNA (partially purified RNA, essentially pure RNA, synthetic RNA, recombinantly produced RNA), as well as altered RNA that differs from naturally occurring RNA by the addition, deletion, substitution and/or alteration of one or more nucleotides. Such alterations can include addition of non-nucleotide material, such as to the end(s) of the small RNA or internally (at one or more nucleotides of the RNA). Nucleotides in the RNA molecules of the present invention can also comprise non-standard nucleotides, including non-naturally occurring nucleotides or deoxyribonucleotides. In a preferred embodiment, the RNA molecules contain a 3′ hydroxyl group.

As used herein, “specifically binds” is meant a compound or antibody which recognizes and binds a polypeptide of the invention but that does not substantially recognize and bind other molecules in a sample, for example, a biological sample, which naturally includes a polypeptide of the invention. In one example, an antibody that specifically binds a STAT3 polypeptide but does not bind a non-STAT3 polypeptide.

As used herein a “small molecule” is an organic compound or such a compound complexed with an inorganic compound (e.g. metal) that has biological activity and is not a polymer. A small molecule generally has a molecular weight of less than about 3 kilodaltons.

The term “antibody” as used herein includes intact molecules such as an immunoglobulin as well as fragments thereof, such as Fab and F(ab′).sub.2, which are capable of binding an epitopic determinant. Antibodies that bind the genes or gene products of the present invention can be prepared using intact polynucleotides or polypeptides or fragments containing small peptides of interest as the immunizing antigen attached to a carrier molecule. The coupled peptide is then used to immunize the animal (e.g, a mouse, rat or rabbit). The antibody may be a “chimeric antibody”, (See, e.g., Cabilly et al., U.S. Pat. No. 4,816,567; and Boss et al., U.S. Pat. No. 4,816,397.). The antibody may be a human or a humanized antibody. The antibody may be a polyclonal, monoclonal, a nanobody. Also included are secondary antibodies.

A “pharmaceutical composition” is intended to include the combination of an active agent with a carrier, inert or active, making the composition suitable for diagnostic or therapeutic use in vitro, in vivo or ex vivo. As used herein, the term “pharmaceutically acceptable carrier” encompasses any of the standard pharmaceutical carriers, such as a phosphate buffered saline, water, diluent, adjuvant, excipient, vehicle and emulsions, such as an oil/water or water/oil emulsion, and various types of wetting agents. The compositions also can include stabilizers and preservatives. Suitable pharmaceutical carriers are described for example by E. W. Martin, REMINGTON'S PHARM. SCI., 15th Ed. (Mack Publ. Co., Easton (1975)).

The term “modulate” or “modulation”, as used herein, refers to either an increase or a decrease in the expression and/or activity or function of STAT3. Thus, a “modulator of STAT3” is defined as an agent that acts as an agonist or stimulator, which enhances expression and/or activity/function of STAT3, or an antagonist or inhibitor, which decreases expression and/or activity/function of STAT3. The activity or function of STAT3, as described herein, relates primarily to its effects on immune homeostasis. More particularly, the activity or function of STAT3, as described herein, relates to its effects on T_{H}17 development and differentiation in vivo and its effects on T_{H}17 dependent autoimmune diseases.

RNA interference (RNAi) is a mechanism that inhibits gene expression at translation or by hindering the transcription of specific genes. The RNAi process directs the degradation of messenger RNAs homologous to short double-stranded RNAs termed “small interfering RNA” or “siRNA” or “short hairpin RNA” or “shRNA”. RNAi is a vital part of the immune response to viruses and foreign genetic material. Stram Y, Kuzntzova L (2006). “Inhibition of viruses by RNA interference”. Virus Genes 32 (3): 299-306. Methods of preparing siRNAs are known to those skilled in the art and are described, for example, in Reich et al., Mol Vis. 9:210-6 (2003); Gonzalez-Alegre P et al., Ann Neurol. 53:781-7 (2003); Miller et al., Proc Natl Acad Sci USA. (2003); Liu and Erikson, Proc Natl Acad Sci USA. 100:5789-94 (2003); Chi et al., Proc Natl Acad Sci USA. 100:6343-6 (2003); Hall and Alexander, J. Virol. 77:6066-9 (2003), all of which are incorporated by reference in their entirety.

“Antisense” nucleic acids are DNA or RNA molecules that are complementary to at least a portion of a specific mRNA molecule (See Weintraub, Sci. Amer. 262:40-46 (1990); Marcus-Sekura, Nucl. Acid Res, 15: 5749-5763 (1987); Marcus-Sekura Anal. Biochem., 172:289-295 (1988); Brysch et al., Cell Mol. Neurobiol., 14:557-568 (1994)). Oligomers of greater than about fifteen nucleotides and molecules that hybridize to the AUG initiation codon will be particularly efficient. Antisense methods have been used to inhibit the expression of many genes in vitro (Marcus-Sekura, Anal. Biochem., 172:289-295 (1988); Hambor et al., Proc. Natl. Acad. Sci. U.S.A. 85:4010-4014 (1988)) and in situ (Arima et al., Antisense Nucl. Acid Drug Dev. 8:319-327 (1998); Hou et al., Antisense Nucl. Acid Drug Dev. 8:295-308 (1998)). Antisense therapy has also been effective in treating Ebola virus in vivo. Bavari et al., Public Library Sci. Pathogens, (2006).

The present invention provides for compositions and methods for modulation of autoimmune conditions. Examples include autoimmune diseases, which include but are not limited to: multiple sclerosis, Crohn's disease, certain bacterially induced colitis, arthritis, lupus, diabetes, asthma, inflammatory bowel disease, scleroderma and vasculitis.

The compositions for modulation of autoimmune diseases comprise an agent that antagonizes the activity of STAT3. Examples of such agents include: antisense RNA and RNAi such as siRNAs or shRNAs, STAT3 siRNAS or shRNAs conjugated to ligands that specifically target T cells, peptides specific for T cell surface molecules or RNA aptamers specific for T cell surface molecules, T cell tropic gene delivery vectors encoding STAT3 siRNA or shRNA, antibodies and fragments thereof such as antibodies to T cell surface molecules, and small molecules. In one aspect, the composition comprises an antagonist to STAT3 wherein the antagonist is capable of reducing or partially inhibiting or completely inhibiting the activity of STAT3. In another aspect, the composition comprises an inhibitor or antagonist to STAT3 wherein the inhibitor is capable of reducing or partially inhibiting STAT3 signaling.

In accordance with the present invention, anti-STAT3 antibodies may be polyclonal or monoclonal; may be from any of a number of human, non-human eukaryotic, cellular, fungal or bacterial sources; may be encoded by genomic or vector-borne coding sequences; and may be elicited against native or recombinant STAT3 or fragments thereof with or without the use of an adjuvant, all according to a variety of methods and procedures well-known in the art for generating and producing antibodies. Neutralizing or antagonistic antibodies against STAT3 (i.e., those that inhibit biological activity of STAT3) are preferred for therapeutic applications. Examples of such useful antibodies include but are not limited to polyclonal, monoclonal, chimeric, single-chain, and various human or humanized types of antibodies, as well as various fragments thereof, such as Fab fragments and fragments produced from specialized expression systems.

The invention also contemplates kits comprising a composition comprising one or more STAT3 inhibitor including excipient. The kit can further comprise instructions for use, such as dosing regimen.

Methods of Using STAT3 Inhibitor for Autoimmune Diseases

The invention provides for methods for treating an autoimmune condition in an individual by inhibiting or antagonizing STAT3 signaling or activity in the individual. The invention also provides for methods for delaying development of an autoimmune condition in an individual by antagonizing STAT3 signaling or activity in the individual. These methods are practiced by administering to an individual a composition comprising an effective amount of STAT3 antagonist to inhibit or reduce STAT3 signaling or its activity. For treatment, the composition comprising an effective amount of STAT3 antagonist can be administered as a pharmaceutical composition by including a pharmaceutically acceptable excipient. The administration can be once or repeatedly over a period of time or as needed as dictated by the appearance of symptoms associated with autoimmune disease. A physician or one of skill in the art can monitor the individual for progress during the course of the treatment.

The compositions may be administered to an individual either alone or admixed with suitable carriers and excipients. The compositions may be administered parenterally, intraperitoneally, subcutaneously, or intramuscularly.

In still other embodiments, the compositions may be administered topically, such as by skin patch. For example, the compositions may be formulated into topical creams, skin or mucosal patches, liquids or gels suitable for topical application to skin or mucosal membrane surfaces. In yet other embodiments, the compositions may be administered by inhaler to the respiratory tract for local or systemic treatment.

The invention also provides for methods of delaying development of autoimmune disease by administration of a composition comprising an effective amount of STAT3 inhibitor. In some cases, the autoimmune disease is delayed or its onset prevented. In other cases, the administration of the STAT3 inhibitor ameliorates one or more symptoms associated with autoimmune disease.

Because of the requirement for IL-6 in T_{H}17 T cell development, a number of laboratories have suggested that STAT3 signaling is involved in the in vitro differentiation of naïve CD4 T cells to a T_{H}17 phenotype (11-13). However, an in vivo requirement for STAT3 in T_{H}17 mediated autoimmunity has not yet been described. In the present invention, T cell targeted STAT3 KO is utilized to show for the first time that STAT3 signaling is absolutely required for T_{H}17 T cell differentiation in vivo in a number of autoimmune models. In addition, STAT3 is required for the maintenance of endogenous, gut-resident, T_{H}17 cells. In these models, promotion of IL-23/T_{H}17 immunity by STAT3 occurs at the expense of IL-12/T_{H}1 immunity. Message-level expression of the transcription factor RORγt appears to occur downstream of STAT3 signaling, as this expression is virtually absent in CD4 T cells that lack STAT3 signaling (14). Taken together, these findings demonstrate that STAT3 signaling is a central component of T_{H}17-dependent autoimmune processes and is a target for therapeutic intervention in autoimmune disease.

Diagnostic Methods

STAT3 levels or STAT3 biological activity can also be used for the diagnosis of autoimmune disease in a subject, or a subject who has a propensity or an increased risk of developing an autoimmune disease.

Alterations in the expression or biological activity of STAT3 in a test sample as compared to a normal reference can be used to diagnose autoimmune disease or a propensity to develop an autoimmune disease.

A subject having an autoimmune disease, or a propensity to develop an autoimmune disease, will show an alteration (e.g., an increase or a decrease of 10%, 20%, 30%, 40%, 50%, 60%, 70%, 80%, 90%, or more) in the expression or biological activity of STAT3 in a subject sample as compared to a normal reference is indicative of an autoimmune disease or a risk of developing the same. The STAT3 polypeptide can include full-length polypeptide, degradation products, alternatively spliced isoforms of the polypeptide, enzymatic cleavage products of the polypeptide, the polypeptide bound to a substrate or ligand, or free (unbound) forms of the polypeptide. In one example, a decrease in the level or biological activity of STAT3 polypeptide or STAT3 nucleic acid in a subject sample as compared to a normal reference sample is indicative of an autoimmune disease or a risk of developing the same.

Standard methods may be used to measure polypeptide levels in any bodily fluid, including, but not limited to, urine, blood, serum, plasma, saliva, or cerebrospinal fluid. Such methods include immunoassay, ELISA, Western blotting using antibodies directed to a STAT3 polypeptide, and quantitative enzyme immunoassay techniques.

The measurement of antibodies specific to a STAT3 polypeptide may also be used for the diagnosis of an autoimmune disease or a propensity to develop the same. Antibodies specific STAT3 may be measured in any bodily fluid, including, but not limited to, urine, blood, serum, plasma, saliva, or cerebrospinal fluid.

Nucleic acid molecules encoding a STAT3 sequence, or fragments or oligonucleotides thereof that hybridize to a nucleic acid molecule encoding a STAT3 sequence at high stringency may be used as a probe to monitor expression of nucleic acid levels of STAT3 in a sample for use in the diagnostic methods of the invention.

Diagnostic methods can include measurement of absolute levels of STAT3 polypeptide, nucleic acid, or antibody, or relative levels of STAT3 polypeptide, nucleic acid, or antibody as compared to a reference sample. In one example, an increase in the level or biological activity of STAT3 polypeptide, nucleic acid, or antibody as compared to a normal reference, is considered a positive indicator of an autoimmune disease or a propensity to develop the same.

In any of the diagnostic methods, the level of a STAT3 polypeptide, nucleic acid, or antibody, or any combination thereof, can be measured at least two different times from the same subject and an alteration in the levels (e.g., by 10%, 20%, 30%, 40%, 50%, 60%, 70%, 80%, 90%, or more) over time is used as an indicator of an autoimmune disease, or the propensity to develop the same. The diagnostic methods that include comparing of the STAT3 polypeptide, nucleic acid, or antibody level to a reference level, such as for example, a prior sample taken from the same subject, a change over time (e.g., an increase in STAT3 polypeptide, nucleic acid or antibody) with respect to the baseline level can be used as a diagnostic indicator of an autoimmune disease, or a predisposition to develop the same. The level of the STAT3 polypeptide, nucleic acid encoding the polypeptide, or antibody that binds the polypeptide in a bodily fluid sample of a subject having an autoimmune disease, or the propensity to develop such a condition may be altered, e.g., increased by as little as 10%, 20%, 30%, or 40%, or by as much as 50%, 60%, 70%, 80%, or 90% or more, relative to the level of the polypeptide, nucleic acid, or antibody in a prior sample or samples.

The invention also provides for a non-human animal model for autoimmune disease comprising the conditional deletion of STAT3 expression in CD4 population of T-cells. The conditionally deleted STAT3^{-/-} CD4 mice have a significantly reduced expression of STAT3 in the endogenous CD4 population of T-cells. Preferably, the animals have a complete functional loss or absence of STAT3 expression or STAT3 activity in the CD4 population of T-cells. The loss of STAT3 expression in the CD4 population also leads to a significant reduction or loss of T_{H}17 cells and/or an increase in the population of T_{H}1 cells as compared to wild-type animals of the same species. The non-human animal of the present invention can be used as an animal model for the testing and/or study of autoimmune disease. The animal model can also be used for the testing, screening and identification of therapeutic or diagnostic methods for the treatment and/or prevention of autoimmune disease.

The following examples are merely illustrative of the present invention and should not be construed as limiting the scope of the invention in any way as many variations and equivalents that are encompassed by the present invention will become apparent to those skilled in the art upon reading the present disclosure.

### EXAMPLES

### Example 1

### Materials and Methods

Mice. CD4-Cre mice (Taconic) were bred to STAT3-flox mice and were on a C57BL/6 background for the EAE experiments. For the autoimmune pneumonitis model, all mouse strains (C3HA^{High} 137 Strain, 6.5 TCR Transgenics, and CD4-Cre×STAT3-flox) were backcrossed greater than ten generations to the B10.D2 background (Jackson Laboratory).

In vitro activation of T cells. Naïve CD62L⁺CD4⁺ T cells were enriched with Miltenyi isolation kit (130-091-751). For determination of STAT3 KO 2×10⁶ T cells were activated with PMA (50 ng/mL) and ionomycinin (500 ng/mL) for 1 hour in the presence of IL-6 (20 ng/mL). For cytokine analysis, cells were activated with Dynabeads® T Cell Expander in the presence of indicated cytokines.

Isolation of gut associated lymphocytes. Lymphocytes were isolated from the lamina propria, gut epithelial layer, and Peyer's patches as previously published (14). Lymphocytes were stimulated in vitro for 4 hours in the presence of PMA, ionomycin, and Brefeldin A (10 μg/mL) followed by intracellular staining for cytokines and flow cytometry.

EAE induction. Mice were induced with MOG 35-55 peptide and CFA, and indicated with 350 μg of Pertussis toxin twice. Clinical scoring was as follows: (0.5) partial tail weakness, (1.0) complete tail paralysis, (1.5) complete tail paralysis with awkward gait, (2.0) complete tail paralysis with moderate hind limb weakness, (2.5) complete tail paralysis with severe hind limb weakness, (3.0) complete hind limb paralysis, (3.5) complete hind limb weakness with forelimb deficits, (4.0) complete tetraplegia, (5.0) dead or moribund. Tissues were harvested from mice on day 22 post-induction.

Autoimmune Pneumonitis. 5×10⁶ 6.5TCR⁺CD4⁺ T cells from wild type (WT) or CD4-Cre STAT3flox 6.5TCR transgenic mice were adoptively transferred into C3HA mice (8-10 weeks) as previously described. Non-transgenic mice were also adoptively transferred with T cells. Vaccinated mice were given 10⁶ pfu of Vaccinia-HA. For flow cytometric assays, animals were harvested on day 4 post adoptive transfer. Cytokine blockade was accomplished using anti-IL-17 and anti-IL-23R, or appropriate isotype controls (R&D Systems), given at a dose of 0.5 mg (i.v.) at the time of adoptive transfer, and an additional 0.5 mg (i.p.) on day 2 post-adoptive transfer.

Quantitative PCR. Total RNA was extracted by RNeasy kit (Qiagen) and cDNA was synthesized. All primers were obtained from Applied Biosystems, except for RORγt (14).

### Example 2

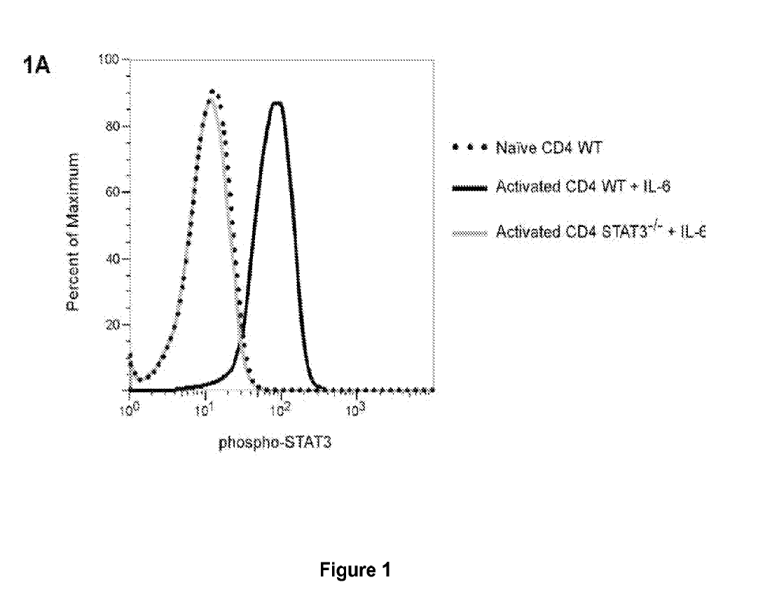

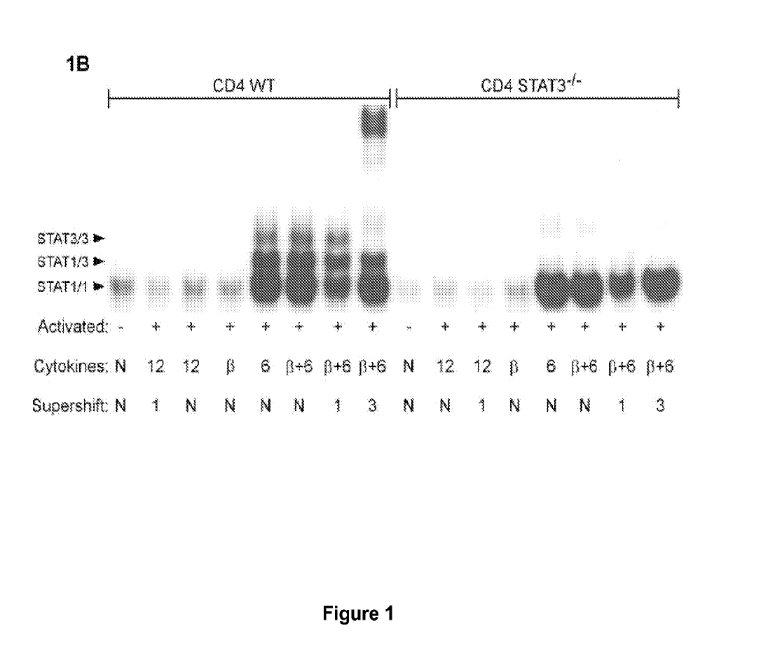

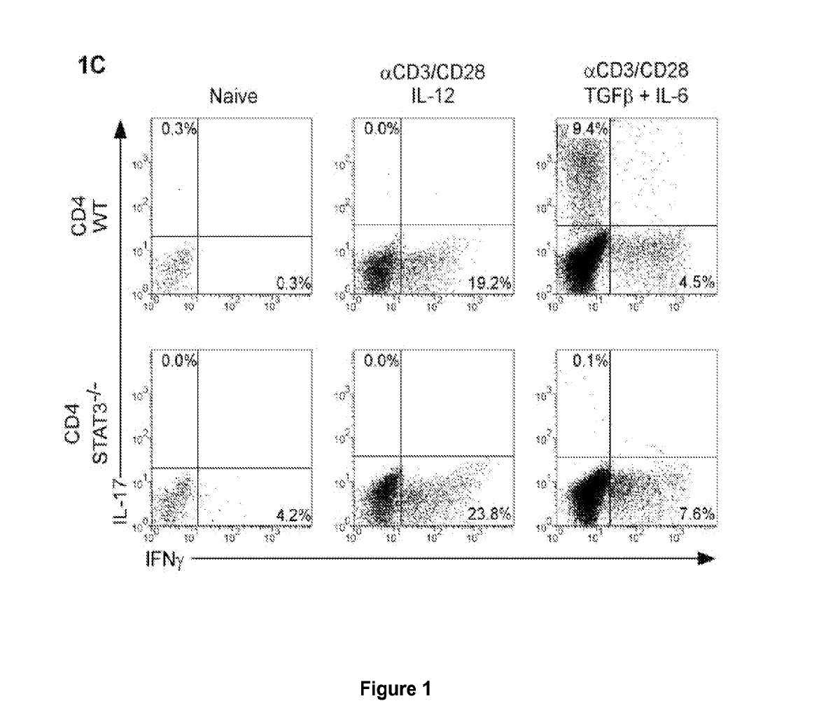

Generation of mice with STAT3 deficient CD4 T cells. Because global STAT3 deficiency is embryonic lethal, STAT3 was conditionally deleted in CD4⁺ T cells by crossing CD4-Cre mice to STAT3^{flox/flox} mice. The CD4-Cre⁺ STAT3^{flox/flox} mice resulting from an F₁-intercross are hereafter referred to as STAT3^{-/-} CD4 mice. To confirm functional STAT3 KO in the CD4 T cell compartment, flow cytometry was performed using anti-phosphoSTAT3 antibodies. CD4⁺ T cells from STAT3^{+/+} CD4 (WT) and STAT3^{-/-} CD4 mice were analyzed following activation in the presence or absence of IL-6. Activated (phosphorylated) STAT3 was detected in CD4 T cells from WT mice, but not in CD4 T cells from the STAT3^{-/-} CD4 animals (FIG. 1a). These data were confirmed by EMSA (FIG. 1b). Both the STAT3-STAT3 homodimer and the STAT3-STAT1 heterodimer were clearly absent in CD4 T cells from the KO animals treated either with IL-6 or with IL-6 in combination with TGF-β; the anti-STAT3 supershift band was also absent in the KO T cells. Taken together, these data confirm functional knockdown of STAT3 in CD4 T cells from STAT3^{-/-} CD4 mice.

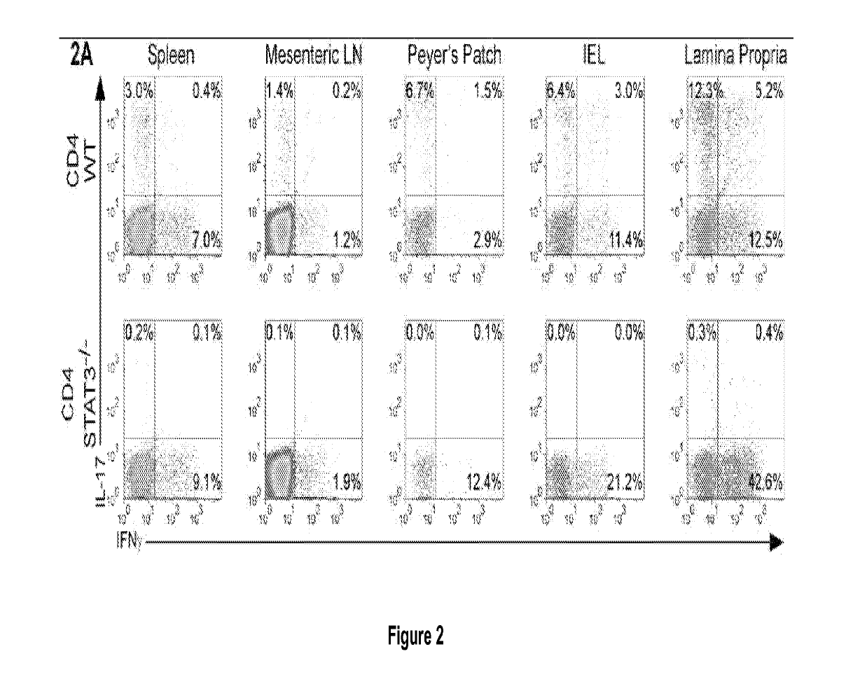

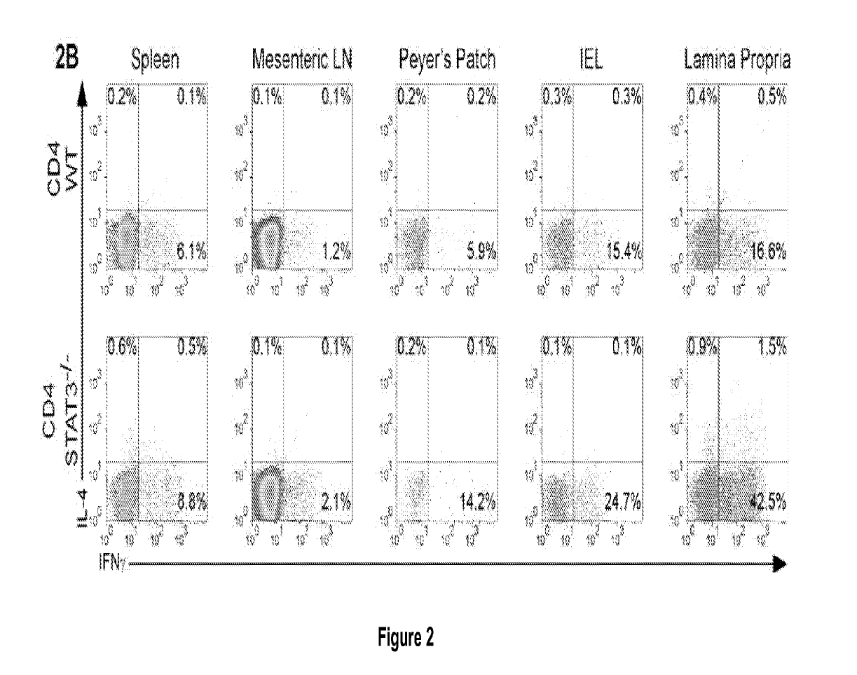

Endogenous T_{H}17 are absent in STAT3^{-/-} CD4 mice. As shown in FIG. 1c genetic KO of STAT3 blocks T_{H}17 development in vitro. To explore the role of STAT3 in T_{H}17 development in vivo, the GALT in WT and STAT3^{-/-} CD4 mice was examined first. Previous work showed that endogenous T_{H}17 T cells are abundant in these tissues in the absence of autoimmune disease (14). As shown in FIG. 2a, T_{H}17 T cells were strikingly absent from all examined compartments in STAT3^{-/-} CD4 mice. This difference was most apparent in lymphocytes isolated from the lamina propria, but was consistent for all other populations studied. The complete loss of T_{H}17-skewed CD4 lymphocytes in STAT3^{-/-} CD4 mice was accompanied by a marked increase in IFN-γ producing cells. This T_{H}1 redirection was most pronounced in the lamina propria, where an ˜3.5-fold increase in the frequency of CD4 T cells secreting IFN-γ was observed. Notably, STAT3 KO did not appear to affect the percentages of CD4 T cells with a T_{H}2 phenotype in vivo (FIG. 2b). Collectively these data demonstrate that STAT3 is required for endogenous, T_{H}17 development in vivo, demonstrating that STAT3 signaling restrains T_{H}1 development.

### Example 3

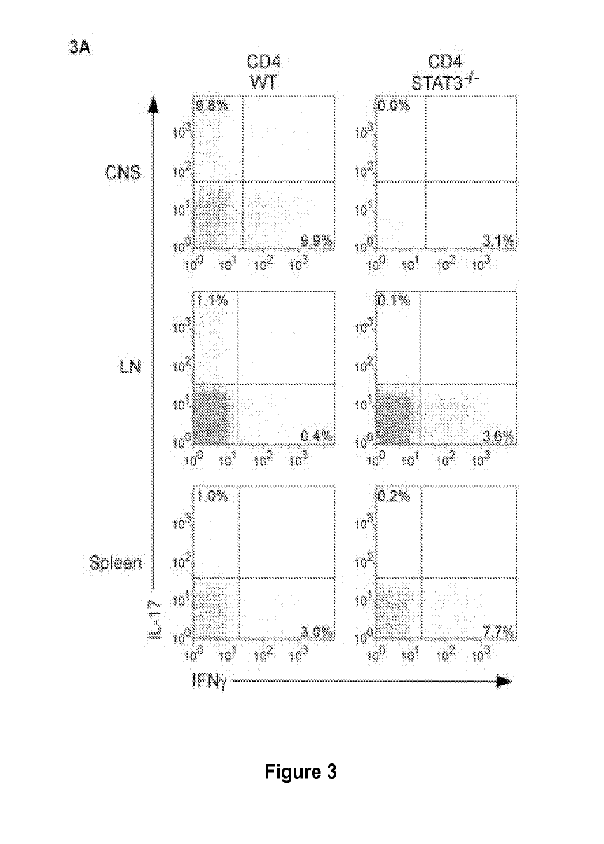

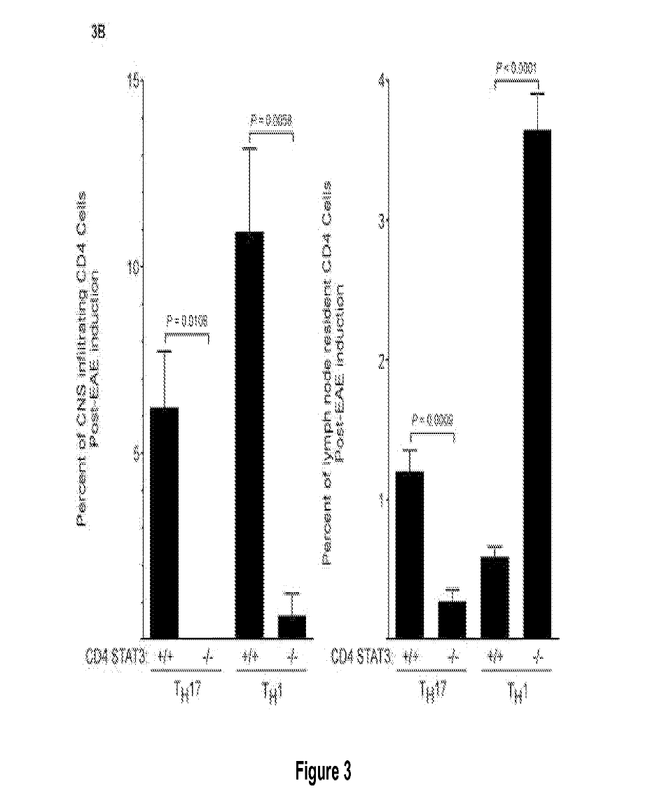

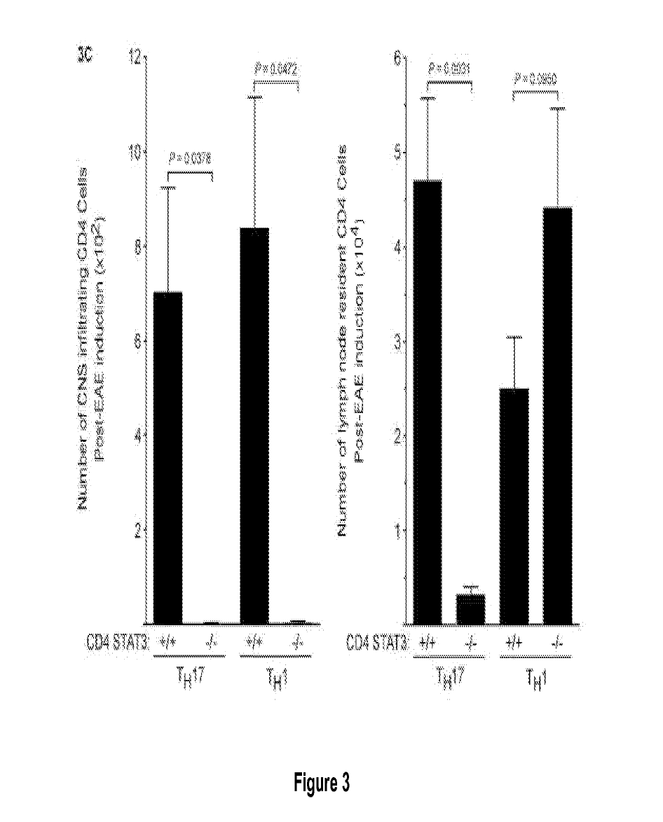

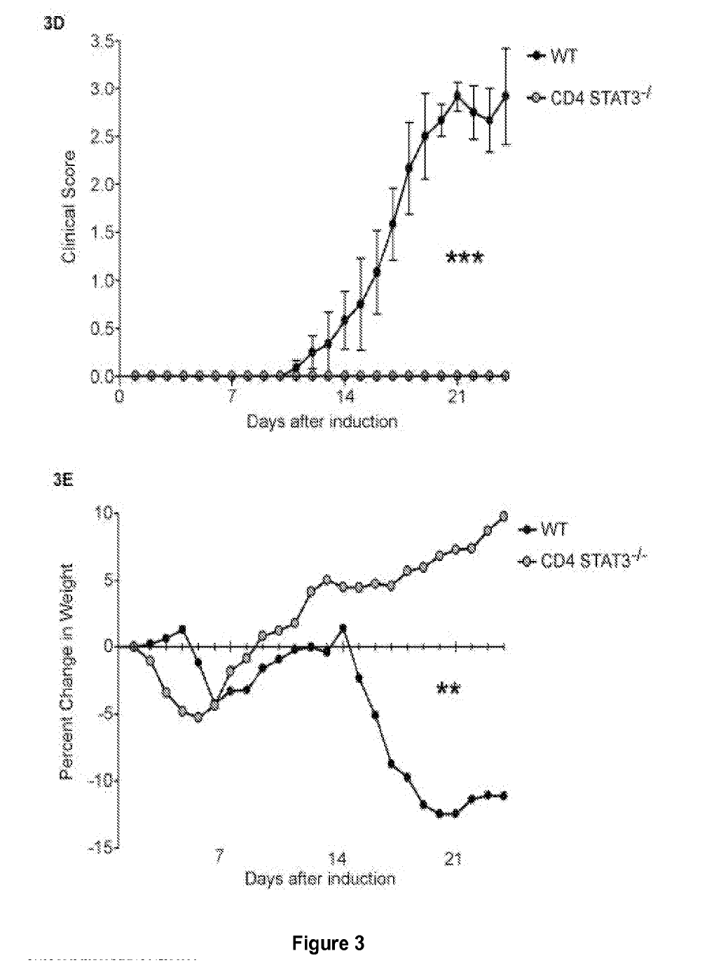

Experimental autoimmune encephalomyelitis is mitigated in STAT3^{-/-} CD4 mice. Considerable data show that the induction of EAE is critically dependent on T_{H}17 T cell differentiation (7, 8). To test whether CD4 T cell expression of STAT3 is required in vivo for disease development, EAE was induced in WT or STAT3^{-/-} CD4 mice. FACS analysis of CNS infiltrating lymphocytes demonstrated that WT mice have an abundant T_{H}17 infiltrate, while STAT3^{-/-} CD4 mice have a paucity of cells in the CNS, with absent T_{H}17 cells and very few T_{H}1 cells (FIG. 3a-c). To address the possibility that the scarcity of CNS-infiltrating CD4 cells in the STAT3^{-/-} CD4 mice was the result of a T cell priming defect, the draining lymph nodes were analyzed from WT or STAT3^{-/-} CD4 mice. Both groups demonstrated a robust response which was grossly evident by enlarged lymph nodes (data not shown) and confirmed by an increase in effector cytokine production (FIG. 3a). However, STAT3^{-/-} CD4 cells produced virtually no IL-17 and robust IFN-γ, demonstrating T_{H}1 skewing in vivo. These data are in sharp contrast to those observed in WT animals, which showed a T_{H}17 skewed phenotype (FIG. 3b, c). The vigorous T_{H}1 immune response in the draining lymph nodes of STAT3^{-/-} CD4 mice effectively excludes a general priming defect of STAT3 deficient CD4 cells, and provides additional in vivo evidence that STAT3 deletion results in an immune response that is skewed away from a T_{H}17 phenotype, and toward a T_{H}1 phenotype in vivo. Strikingly, CD4 T cell expression of STAT3 was absolutely required for disease progression (FIG. 3d). STAT3^{-/-} CD4 mice had a negligible disease score and continued to gain weight through the experimental course (FIG. 3d, e). These data indicate that the development of pathogenic T_{H}17 cells in vivo is dependent on STAT3 signaling, and that abrogation of this signaling pathway results in mitigation of autoimmune disease progression.

### Example 4

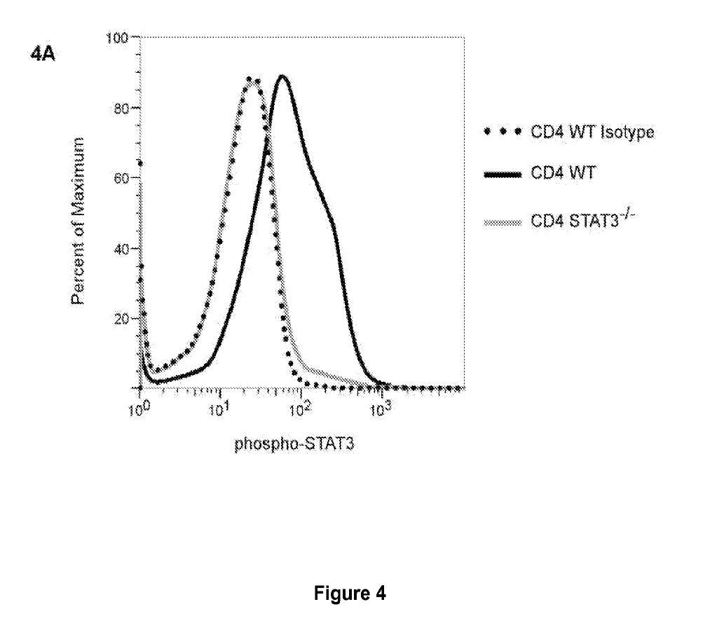

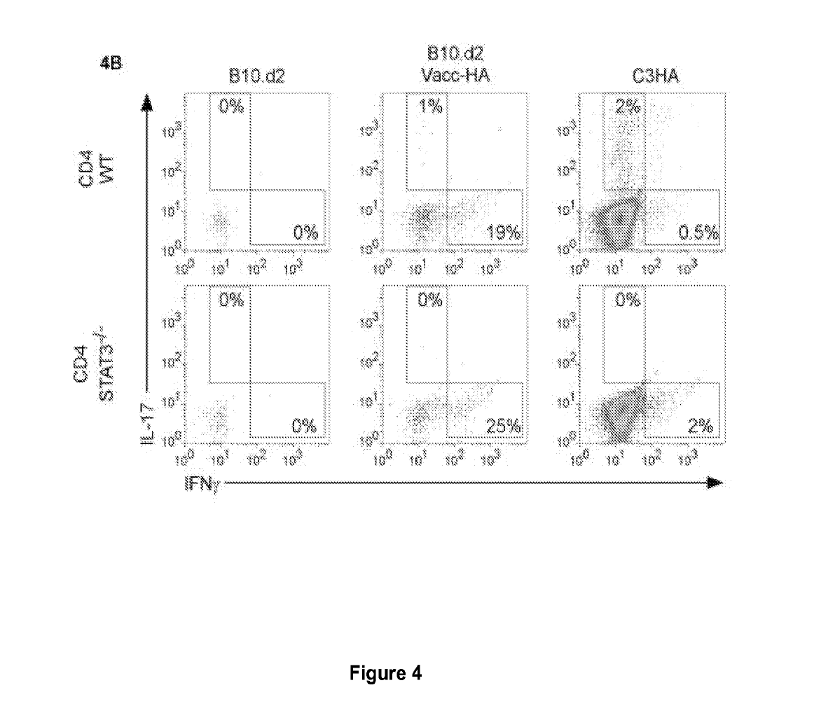

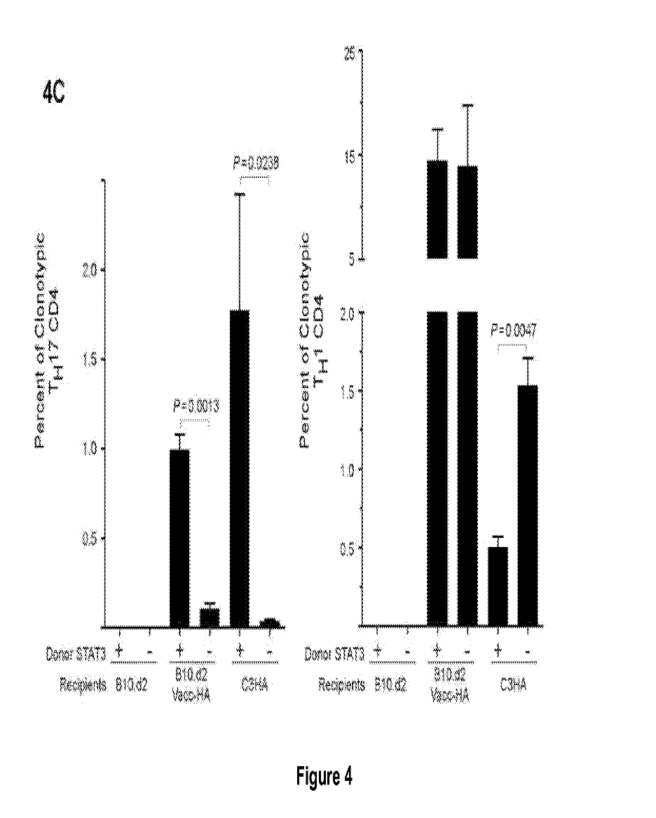

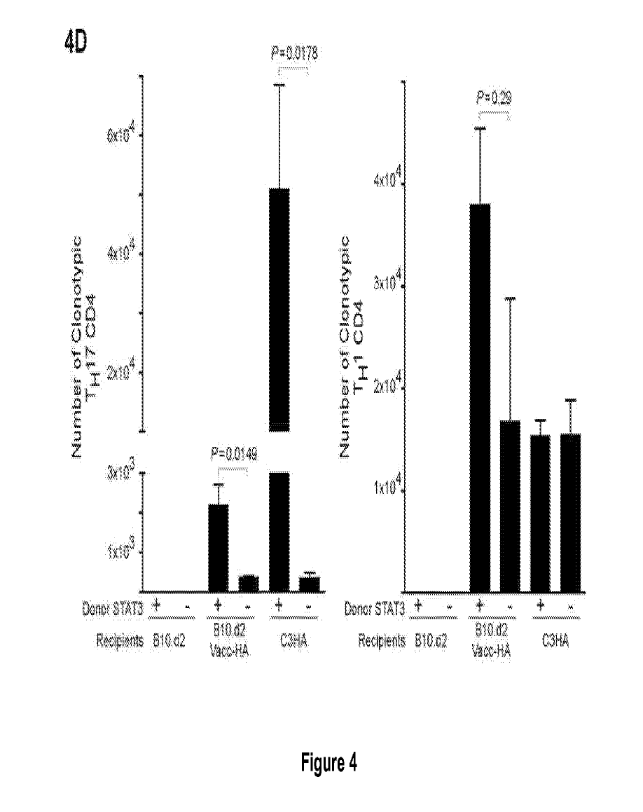

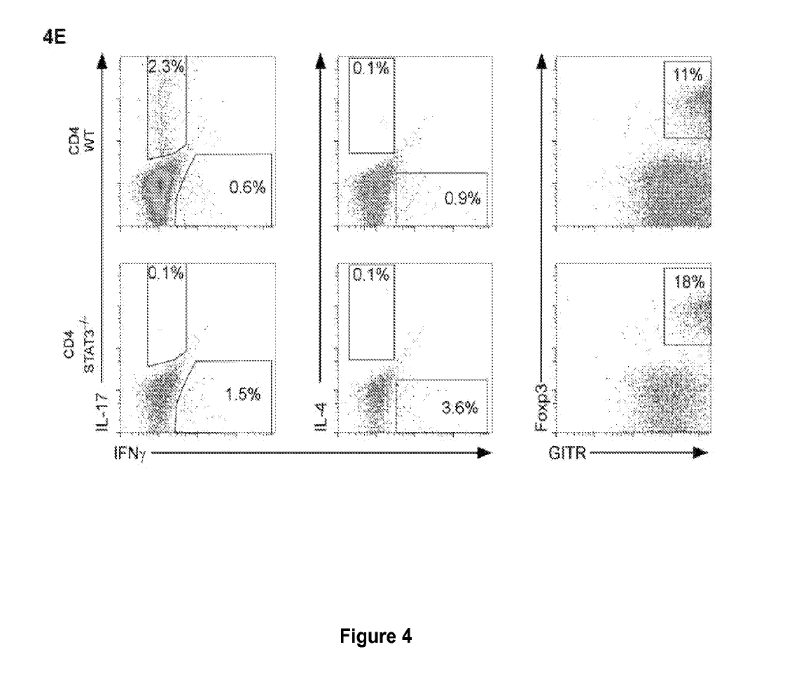

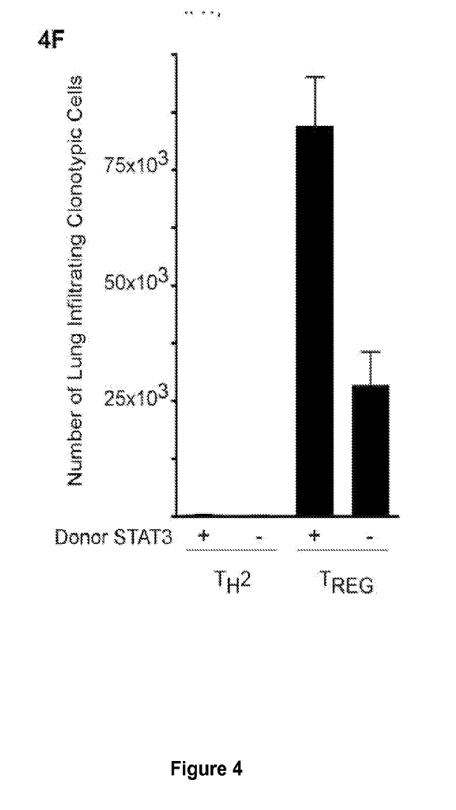

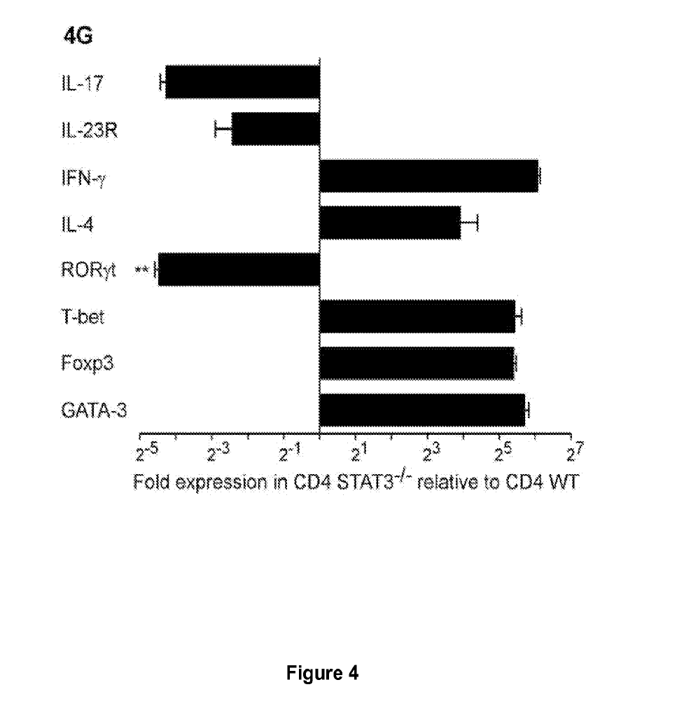

STAT3 signaling is required for T_{H}17-dependent autoimmune pneumonitis. A number of experimental models of autoimmunity have recently been shown to depend on IL-23 and T_{H}17 CD4 responses in vivo (15-17). To further examine a potential role for STAT3 blockade in autoimmunity, a model of induced autoimmune pneumonitis was examined. Adoptive transfer of naïve, HA-specific CD4 T cells from TCR transgenic donors into recipient mice that express HA as a “self-antigen” in the lung (C3HA) results in a fatal autoimmune pneumonitis (18, 19). To determine a role for STAT3 in this process, these TCR transgenic donors were backcrossed onto the STAT3^{-/-} CD4 background, and adoptively transferred T cells from WT and STAT3^{-/-} CD4 donors to C3HA recipients. WT TCR transgenic CD4 T cells recovered from recipient lungs showed an upregulation of activated STAT3 after adoptive transfer, while activated STAT3 could not be detected in T cells isolated from C3HA recipient mice that received T cells from STAT3^{-/-} TCR transgenic donors (FIG. 4a). WT TCR transgenic T cells were skewed towards a T_{H}17 phenotype in C3HA mice (FIG. 4b). T_{H}1 cells were significantly less prominent, with <1% of the HA-specific WT T cells staining positive for IFN-γ (FIG. 4b). This skewing contrasted markedly with the response to infection with a virus, Vaccinia-HA, which is characterized by a dominant T_{H}1 pattern (FIG. 4b-d). The cytokine profile from HA-specific STAT3^{-/-} CD4 cells showed a striking deficit in IL-17 production, and a relative compensatory skewing towards T_{H}1 in C3HA mice (FIG. 4b-d). Under viral-activating conditions, neither WT nor STAT3^{-/-} CD4 cells produced appreciable levels of IL-17 (FIG. 4b-d), and the relative skewing of the STAT3^{-/-} CD4 towards a T_{H}1 phenotype was less dramatic (FIG. 4c). These data show that, similar to several other autoimmune models, T_{H}17 T cells play a more significant role in the observed immunopathology than IFN-γ secreting T_{H}1 cells.

Whether the loss of STAT3 signaling altered differentiation to T_{H}2 and regulatory T cell (T_{REG}) subtypes was determined. Neither WT nor STAT3^{-/-} CD4 cells secreted appreciable quantities of IL-4 after brief ex vivo stimulation (FIG. 4e). There was a modest increase in the frequency of T_{REG} cells in the CD4 population derived from STAT3^{-/-} donors. This increased frequency was not reflected in total T_{REG} cell numbers (FIG. 4f), suggesting that the major consequence of STAT3 KO in this model of autoimmunity was a relative increase in T_{H}1 responses. To more completely assay the effects of STAT3 KO on T cell skewing upon self-antigen recognition, lung-infiltrating TCR transgenic CD4 cells were sorted and Quantitative RT-PCR was performed (FIG. 4g). STAT3 KO resulted in a net deviation away from the T_{H}17 phenotype with complete loss of detectable RORγt and large decreases in IL-17 and IL-23R, while conversely, the STAT3^{-/-} CD4 T cells showed message-level increases in the canonical transcription factors associated with T_{REG}, T_{H}1, and T_{H}2 subtypes. These results for RORγt, IL-17, IL-23R, IFN-γ and T-bet correlate well with the intracellular cytokine staining for IL-17 and IFN-γ, supporting the conclusions from FIG. 2 that STAT3 signaling drives the T_{H}17 pathway at the expense of the T_{H}1 pathway in vivo. The correlations between Quantitative RT-PCR and staining for T_{H}2 and T_{REG} pathways may reflect low gene expression levels and/or post-transcriptional regulation of protein levels.

### Example 5

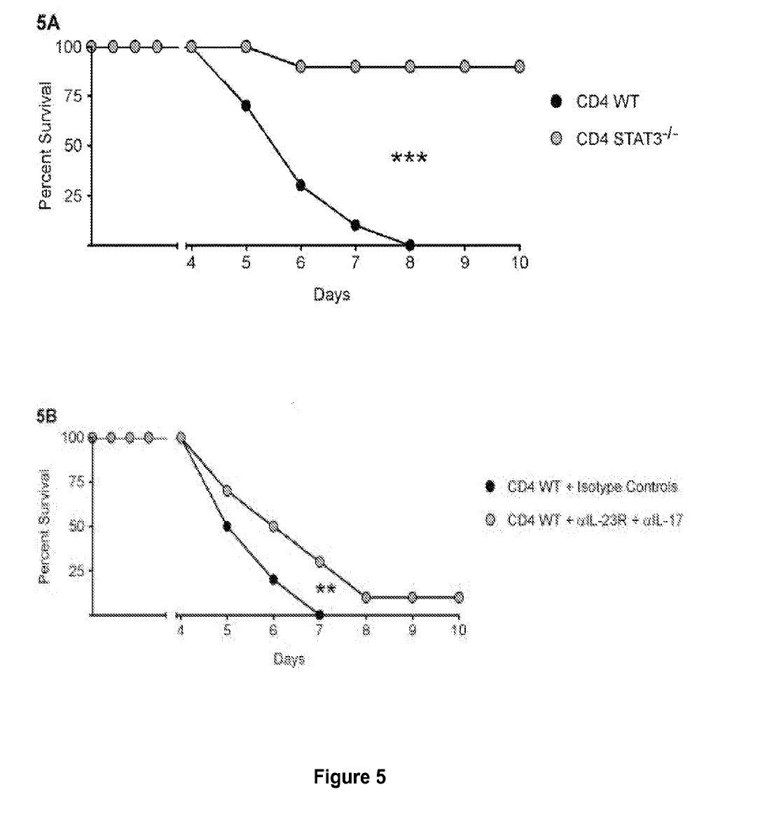

Fatal autoimmune pneumonitis requires STAT3. Whether clinical outcome in the pneumonitis model was directly dependent on STAT3 signaling was determined. STAT3^{-/-} CD4 T cells were strikingly less efficient in promoting autoimmune lethality than WT cells (FIG. 5a). The mortality rate of the WT recipient group was 100%, with a median survival time ranging from 5-6 days; however, the KO recipient group displayed a mortality rate ranging from only 0-10%. To determine whether STAT3 dependent T_{H}17 responses contributed to the autoimmune pneumonitis, C3HA mice were treated with a combination of anti-IL-23R and anti-IL-17 antibodies after an adoptive transfer of WT HA-specific CD4 T cells. FIG. 5b demonstrates that lethal pneumonitis was partially inhibited by in vivo anti-IL-23R/anti-IL-17 treatment. Similar to results in other autoimmunity models such as EAE (8), the intermediate effects of IL23R/IL-17 blockade seen here may reflect partial blockade, or that full blockade is difficult to achieve in the context of a potent, in vivo proliferative autoimmune response.

Taken together, the results demonstrate a requirement for STAT3 signaling in T_{H}17 differentiation in vivo and that STAT3 signaling also mediates a relative inhibition of T_{H}1 differentiation. In vivo, endogenous T_{H}17 cells are found in the lamina propria of the small bowel and these endogenous T_{H}17 cells are not associated with a pathologic state. T_{H}17 cells were completely absent from the GALT of STAT3^{-/-} CD4 mice, similar to what was observed in RORγt KO mice (14). In these studies of endogenous cells, IFN-γ producing T_{H}1 cells were dramatically increased in number, further demonstrating that STAT3 shifts immunity from T_{H}1 to T_{H}17. O'Shea and colleagues provided the first evidence that STAT3 played a role in T_{H}17-related transcription by demonstrating STAT3 binding to the IL-17 promoter (21). These in vivo studies show that STAT3 plays a broad role in T_{H} skewing by reciprocally regulating master transcription factors for the T_{H}1 and T_{H}17 lineages (T-bet and RORγt respectively).

EAE was one of the first experimental autoimmune models shown to be mediated by the T_{H}17 subset. The data clearly indicate that the appearance of T_{H}17 cells in this experimental model is dependent upon the expression of STAT3 in naïve CD4 T cells. “EAE-induced” CD4 STAT3^{-/-} mice prime their T cells but with a resultant T_{H}1 response. Furthermore, the T_{H}1 cells in CD4 STAT3^{-/-} mice, do not traffic to the CNS, and the few cells that reach this site do not mediate a clinical phenotype. Experimental results from the C3HA autoimmune pneumonitis model were equally striking; STAT3^{-/-} CD4 T cells were unable to mediate lethal pneumonitis. In concordance with cytokine data from our ex vivo stimulation, the lung-infiltrating STAT3^{-/-} CD4 T cells demonstrated increased transcription of T-betsuggestive of immune deviation towards T_{H}1. The finding that blockade of IL-23R and IL-17 partially inhibited lethality in the pneumonitis model confirms a pathophysiologic role for STAT3 driven IL-23/T_{H}17 immunity. The inhibition of lethality with anti-IL-23R/anti-IL-17 blockade was not nearly as complete as with STAT3^{-/-} T cells. This may be due to incomplete blockade by the antibodies or additional effects of STAT3 besides promotion of IL-23/T_{H}17 immunity. This result is consistent with previously published data demonstrating delayed disease kinetics as a result of antibody blockade (8), whereas complete absence of disease is observed in cytokine KO animals (15, 16). The observation that T_{H}1 cells are increased in proportion and maintain absolute numbers after STAT3^{-/-} T cell transfer suggests that T_{H}1 responses are not responsible for the lethal autoimmune pneumonitis observed in this model. Finally, the finding that absolute numbers of T_{REG} cells are somewhat decreased after STAT3^{-/-} T cell transfer argues against an enhanced protective effect of T_{REG} cells to explain the abrogation of lethality in the absence of STAT3. A recent report demonstrated that IL-22, another T_{H}17 cytokine whose receptor activates STAT3, is responsible for IL-23-dependent dermal acanthosis (22).

T_{H}17 cells have been positively correlated to the pathophysiology of autoimmune diseases, including rheumatoid arthritis and multiple sclerosis in experimental models, as well as in humans (23, 24). As many current therapies for autoimmune disease are limited by nonspecific immunosupression, STAT3 serves as a novel target for autoimmunity, since genetic downregulation of STAT3 in CD4 T cells results in an absence of T_{H}17 T cells accompanied by a relative augmentation of the T_{H}1 response in vivo.

### Example 6

### Antagonist of STAT3 for Treating or Delaying Development of Autoimmune Diseases

Antagonists to STAT3, such as small molecule inhibitors of Stat3, Stat3 siRNAs or shRNAs, STAT3 siRNAs or shRNAs conjugated to ligands that specifically target T cell, peptides specific for T cell surface molecules or RNA aptamers specific for T cell surface molecules, T cell tropic gene delivery vectors, antibodies and fragments thereof are used for inhibiting STAT3 activity or STAT3 signaling. Administration or delivery of the antagonists results in the prevention or delay in the development of T_{H}17 cells. In some cases, there is a partial inhibition of the development of Th17 cells. In other cases, there is a complete inhibition of the development of Th17 cells.

### REFERENCES

1. Krause, A., N. Scaletta, J. D. Ji, and L. B. Ivashkiv. 2002. Rheumatoid arthritis synoviocyte survival is dependent on Stat3. J Immunol 169:6610-6616.
2. Jee, Y., G. Kim, N. Tanuma, and Y. Matsumoto. 2001. STAT expression and localization in the central nervous system during autoimmune encephalomyelitis in Lewis rats. J Neuroimmunol 114:40-47.
3. Frisullo, G., F. Angelucci, M. Caggiula, V. Nociti, R. Iorio, A. K. Patanella, C. Sancricca, M. Mirabella, P. A. Tonali, and A. P. Batocchi. 2006. pSTAT1, pSTAT3, and T-bet expression in peripheral blood mononuclear cells from relapsing-remitting multiple sclerosis patients correlates with disease activity. J Neurosci Res 84:1027-1036.
4. Liu, K., C. Liang, Z. Liang, K. Tus, and E. K. Wakeland. 2005. Sle1ab mediates the aberrant activation of STAT3 and Ras-ERK signaling pathways in B lymphocytes. J Immunol 174:1630-1637.
5. Bao, L., J. U. Lindgren, P. van der Meide, S. Zhu, H. G. Ljunggren, and J. Zhu. 2002. The critical role of IL-12p40 in initiating, enhancing, and perpetuating pathogenic events in murine experimental autoimmune neuritis. Brain Pathol 12:420-429.
6. Oppmann, B., R. Lesley, B. Blom, J. C. Timans, Y. Xu, B. Hunte, F. Vega, N. Yu, J. Wang, K. Singh, F. Zonin, E. Vaisberg, T. Churakova, M. Liu, D. Gorman, J. Wagner, S. Zurawski, Y. Liu, J. S. Abrams, K. W. Moore, D. Rennick, R. de Waal-Malefyt, C. Hannum, J. F. Bazan, and R. A. Kastelein. 2000. Novel p19 protein engages IL-12p40 to form a cytokine, IL-23, with biological activities similar as well as distinct from IL-12. Immunity 13:715-725.
7. Langrish, C. L., Y. Chen, W. M. Blumenschein, J. Mattson, B. Basham, J. D. Sedgwick, T. McClanahan, R. A. Kastelein, and D. J. Cua. 2005. IL-23 drives a pathogenic T cell population that induces autoimmune inflammation. J Exp Med 201:233-240.
8. Park, H., Z. Li, X. O. Yang, S. H. Chang, R. Nurieva, Y. H. Wang, Y. Wang, L. Hood, Z. Zhu, Q. Tian, and C. Dong. 2005. A distinct lineage of CD4 T cells regulates tissue inflammation by producing interleukin 17. Nat Immunol 6:1133-1141.
9. Veldhoen, M., R. J. Hocking, C. J. Atkins, R. M. Locksley, and B. Stockinger. 2006. TGFbeta in the context of an inflammatory cytokine milieu supports de novo differentiation of IL-17-producing T cells. Immunity 24:179-189.
10. Bettelli, E., Y. Carrier, W. Gao, T. Korn, T. B. Strom, M. Oukka, H. L. Weiner, and V. K. Kuchroo. 2006. Reciprocal developmental pathways for the generation of pathogenic effector TH17 and regulatory T cells. Nature 441:235-238.
11. Yang, X. O., A. D. Panopoulos, R. Nurieva, S. H. Chang, D. Wang, S. S. Watowich, and C. Dong. 2007. STAT3 regulates cytokine-mediated generation of inflammatory helper T cells. J Biol Chem.
12. Laurence, A., C. M. Tato, T. S. Davidson, Y. Kanno, Z. Chen, Z. Yao, R. B. Blank, F. Meylan, R. Siegel, L. Hennighausen, E. M. Shevach, and J. O'Shea J. 2007. Interleukin-2 signaling via STAT5 constrains T helper 17 cell generation. Immunity 26:371-381.
13. Mathur, A. N., H. C. Chang, D. G. Zisoulis, G. L. Stritesky, Q. Yu, J. T. O'Malley, R. Kapur, D. E. Levy, G. S. Kansas, and M. H. Kaplan. 2007. Stat3 and Stat4 direct development of IL-17-secreting Th cells. J Immunol 178:4901-4907.
14. Ivanov, II, B. S. McKenzie, L. Zhou, C. E. Tadokoro, A. Lepelley, J. J. Lafaille, D. J. Cua, and D. R. Littman. 2006. The orphan nuclear receptor RORgammat directs the differentiation program of proinflammatory IL-17+ T helper cells. Cell 126:1121-1133.
15. Cua, D. J., J. Sherlock, Y. Chen, C. A. Murphy, B. Joyce, B. Seymour, L. Lucian, W. To, S. Kwan, T. Churakova, S. Zurawski, M. Wiekowski, S. A. Lira, D. Gorman, R. A. Kastelein, and J. D. Sedgwick. 2003. Interleukin-23 rather than interleukin-12 is the critical cytokine for autoimmune inflammation of the brain. Nature 421:744-748.
16. Murphy, C. A., C. L. Langrish, Y. Chen, W. Blumenschein, T. McClanahan, R. A. Kastelein, J. D. Sedgwick, and D. J. Cua. 2003. Divergent pro- and antiinflammatory roles for IL-23 and IL-12 in joint autoimmune inflammation. J Exp Med 198:1951-1957.
17. Yen, D., J. Cheung, H. Scheerens, F. Poulet, T. McClanahan, B. McKenzie, M. A. Kleinschek, A. Owyang, J. Mattson, W. Blumenschein, E. Murphy, M. Sathe, D. J. Cua, R. A. Kastelein, and D. Rennick. 2006. IL-23 is essential for T cell-mediated colitis and promotes inflammation via IL-17 and IL-6. J Clin Invest 116:1310-1316.
18. Huang, C. T., C. J. Workman, D. Flies, X. Pan, A. L. Marson, G. Zhou, E. L. Hipkiss, S. Ravi, J. Kowalski, H. I. Levitsky, J. D. Powell, D. M. Pardoll, C. G. Drake, and D. A. Vignali. 2004. Role of LAG-3 in regulatory T cells. Immunity 21:503-513.
19. Huang, X., J. Zhu, and Y. Yang. 2005. Protection against autoimmunity in nonlymphopenic hosts by CD4+CD25+ regulatory T cells is antigen-specific and requires IL-10 and TGF-beta. J Immunol 175:4283-4291.
20. Cho, M. L., J. W. Kang, Y. M. Moon, H. J. Nam, J. Y. Jhun, S. B. Heo, H. T. Jin, S. Y. Min, J. H. Ju, K. S. Park, Y. G. Cho, C. H. Yoon, S. H. Park, Y. C. Sung, and H. Y. Kim. 2006. STAT3 and NF-kappaB signal pathway is required for IL-23-mediated IL-17 production in spontaneous arthritis animal model IL-1 receptor antagonist-deficient mice. J Immunol 176:5652-5661.
21. Chen, Z., A. Laurence, Y. Kanno, M. Pacher-Zavisin, B. M. Zhu, C. Tato, A. Yoshimura, L. Hennighausen, and J. J. O'Shea. 2006. Selective regulatory function of Socs3 in the formation of IL-17-secreting T cells. Proc Natl Acad Sci USA 103:8137-8142.
22. Zheng, Y., D. M. Danilenko, P. Valdez, I. Kasman, J. Eastham-Anderson, J. Wu, and W. Ouyang. 2006. Interleukin-22, a T(H)17 cytokine, mediates IL-23-induced dermal inflammation and acanthosis. Nature.
23. Lock, C., G. Hermans, R. Pedotti, A. Brendolan, E. Schadt, H. Garren, A. Langer-Gould, S. Strober, B. Cannella, J. Allard, P. Klonowski, A. Austin, N. Lad, N. Kaminski, S. J. Galli, J. R. Oksenberg, C. S. Raine, R. Heller, and L. Steinman. 2002. Gene-microarray analysis of multiple sclerosis lesions yields new targets validated in autoimmune encephalomyelitis. Nat Med 8:500-508.
24. Chabaud, M., J. M. Durand, N. Buchs, F. Fossiez, G. Page, L. Frappart, and P. Miossec. 1999. Human interleukin-17: A T cell-derived proinflammatory cytokine produced by the rheumatoid synovium. Arthritis Rheum 42:963-970.

1. A method for treating an autoimmune disease in an individual comprising administering to the individual a composition comprising a therapeutically effective amount of an antagonist of STAT3 wherein the antagonist decreases the activity of STAT3 in the individual.

2. The method of claim 1 wherein the autoimmune disease is selected from the group consisting of: multiple sclerosis, rheumatoid arthritis, Crohn's disease, bacterially induced colitis, asthma, inflammatory bowel disease, scleroderma, diabetes, lupus, asthma, and vasculitis.

3. The method of claim 1 wherein the antagonist is selected from the group consisting of a small molecule, an antisense RNA, siRNA, siRNA conjugated to a ligand that specifically targets a T cell, RNA aptamer specific for a T cell surface molecule, and an antibody or fragment thereof.

4. The method of claim 1 wherein the treating results in a decrease in the number of T_{H}17 cells as compared to the number of T_{H}17 cells in the individual prior to treatment.

5. The method of claim 1 wherein treatment with the antagonist results in a reduction of STAT3 activity in the individual.

6. A method of inhibiting T_{H}17 cell differentiation in a mammal comprising administering at least one STAT3 antagonist to the mammal such that there is a reduced number of differentiated T_{H}17 cells in the mammal after STAT3 antagonist administration as compared to the number found in the mammal before STAT3 antagonist administration.

7. The method of claim 6, wherein the antagonist is selected from the group consisting of a small molecule, an antisense RNA, siRNA, siRNA conjugated to a ligand that specifically targets a T cell, a RNA aptamer specific for a T cell surface molecule, and an antibody or fragment thereof.

8. The method of claim 6 wherein the reduced number of differentiated T_{H}17 cells indicates a reduced incidence or intensity of an autoimmune disease.

9. The method of claim 8, wherein the autoimmune disease is autoimmune pneumonitis.

10. The method of claim 6, wherein the T_{H}17 cells measured for the comparison are sampled or obtained from a lamina propria region of tissue of the mammal.

11. A method of disrupting STAT3 signaling comprising administering an effective amount of a STAT3 antagonist to inhibit signaling, wherein the disruption in signaling treats or prevents an autoimmune disease.

12. The method of claim 11, wherein the STAT3 antagonist is selected from the group consisting of a small molecule, an antisense RNA, siRNA, siRNA conjugated to a ligand that specifically targets a T cell, a RNA aptamer specific for a T cell surface molecule, and an antibody or fragment thereof.

13. The method of claim 12, wherein a combination of more than one type of the group of STAT3 inhibitors in administered.

14. The method of claim 13, wherein STAT3 inhibitors are administered in series.

15. The method of claim 11, wherein the autoimmune disease is multiple sclerosis, rheumatoid arthritis, Crohn's disease, bacterially induced colitis, asthma, inflammatory bowel disease, scleroderma, diabetes, lupus, asthma, vasculitis, or pneumonitis.

16. A method of diagnosing a subject as having or having a propensity to develop an autoimmune disease comprising determining the level or biological activity of a STAT3 polypeptide or nucleic acid in a sample from the subject, wherein a greater level or biological activity of the STAT3 polypeptide or nucleic acid in the sample relative to a reference sample or reference level is diagnostic of an autoimmune disease or a propensity to develop an autoimmune disease in the subject.

17. The method of claim 16 wherein the autoimmune disease is multiple sclerosis, rheumatoid arthritis, Crohn's disease, bacterially induced colitis, asthma, inflammatory bowel disease, scleroderma, diabetes, lupus, asthma, vasculitis, or pneumonitis.

18. A non-human animal whose genome comprises a conditional disruption in expression of at least one allele of the STAT3 gene.

19. The animal of claim 18 wherein the disruption results in inhibition of STAT3 expression in the animal's CD4⁺ population of T cells.

20. The animal of claim 19 wherein the disruption leads to one or more characteristic selected from the group consisting of: a) a substantially lower number of endogenous T_{H}17 T cells as compared to a wild-type animal of the same species; b) a substantially higher number of endogenous T_{H}1 cells as compared to a wild-type animal of the same species; c) a substantially lower level of endogenous IL-17 as compared to a wild-type animal of the same species; and substantially lower level of endogenous IL-23R as compared to a wild-type animal of the same species.

21. The animal of claim 18 wherein the animal is a rodent.

22. The animal of claim 21 wherein the animal is a mouse.
